(19) United States
(12) Patent Application Publication
(10) Pub. No.: US 2003/0082604 A1
(43) Pub. Date: May 1, 2003

(54) High density arrays

(76) Inventors: Melvin J. Swanson, Carver, MN (US); Patrick E. Guire, Eden Prairie, MN (US)

Correspondence Address: MERCHANT & GOULD PC, P.O. BOX 2903, MINNEAPOLIS, MN, 55402-0903, US

(21) Appl. No.: 10/233,071

(22) Filed: Aug. 30, 2002

Related U.S. Application Data
(63) Continuation-in-part of application No. 09/670,766, filed on Sep. 27, 2000, now abandoned.

(51) Int. Cl. C12Q 1/68; B05D 3/00; G01N 33/53; G01N 33/542; C12M 1/34

(52) U.S. Cl. 435/6; 435/7.9; 435/287.2; 427/2.11

(57) ABSTRACT

The invention provides a method for generating arrays with a variety of densities, in particular, high density arrays. Generally, the method includes a printing step and an illumination step. In the printing step, a predetermined volume of a reagent solution containing receptor molecules is applied to a solid support in a desired pattern. In one embodiment, the receptor molecule is derivatized with a photoreactive agent. In an alternate embodiment, the solid support includes a photoreactive agent. In a preferred embodiment, the receptor molecule is a nucleic acid. In the illumination step, the photoreactive groups are irradiated to immobilize the receptor molecule to the solid support. In one embodiment, a mask having the same center to center distance (e.g., pitch) as the printed spots, but a smaller diameter, is placed over the printed pattern and illuminated. Preferably the mask illuminates a spot having a smaller diameter than the printed spots. Thus, according to the invention, the immobilized reagent spot has a smaller diameter than the original printed spot. In an alternate embodiment, the illumination step can be carried out using mirrored laser technology. If desired, the application and illumination of offset spots can be repeated to form a high density array.

### RELATED APPLICATIONS

This application is a Continuation-In-Part application of U.S. patent application Ser. No. 09/670,766 filed Sep. 27, 2000, the entire disclosure of which is incorporated herein by reference.

### TECHNICAL FIELD

The present invention relates to the immobilization of nucleic acids onto a solid support. More particularly, the invention relates to high density nucleic acid arrays.

### BACKGROUND OF THE INVENTION

Microarrays are small surfaces (typically 2-3 cm² wafers of silicon or glass slides) on which different nucleic acid sequences are immobilized. Typically, the nucleic acids are immobilized at precise locations on the surface via in situ solid phase synthesis or covalent immobilization of nucleic acids to the surface. The nucleic acids serve as probes for detecting complementary nucleic acid sequences. The array can have from hundreds to thousands of immobilized nucleic acids. A dense array may have more than 1000 nucleic acid sequences per square cm.

To use a microarray, fluorescently labeled DNA or RNA sequences (either synthetic or obtained from a cell of interest) are contacted with the array. The hybridization pattern of the fluorescently labeled fragments can provide a wealth of information.

Microarrays have the unique ability to track the expression of many of a cell's genes at once, allowing researchers to view the behavior of thousands of genes in concert. Thus, arrays are useful for diagnostics. Detection of unique gene expression patterns may assist a physician in pinpointing the onset of diseases such as cancer, Alzheimer's, osteoporosis and heart disease. Arrays are also useful for understanding which genes are active in a particular disease. Arrays are also useful for pathogen identification, forensic applications, monitoring mRNA expression and de novo sequencing. See, for instance, Lipshutz, et al., Bio Techniques, 19(3);442-447(1995).

Microarrays can be manufactured using a variety of techniques. For example, the various oligonucleotides can be manufactured by solid phase synthesis on the array surface. See, for example, PCT Publication No. WO 92/10092 (Affymax Technologies N.V.). Although arrays having relatively high densities can be manufactured by solid phase synthesis, the length of the nucleic acid sequence is limited. With present techniques, it is common that every addition step in the synthesis of nucleic acids will result in some errors or truncated sequences. However, with oligonucleotide microchips prepared by in situ solid phase synthesis, post-synthesis purification techniques (e.g., HPLC) are not possible. Thus, such arrays are generally constructed with relatively short nucleic acid sequences (approx. 20 mers) to limit the amount of error.

Alternately, microarrays can be manufactured by immobilizing pre-existing nucleic acids (e.g., oligonucleotides, cDNAs or PCR products) onto the array surface. For example, Synteni (Palo Alto, Calif.) manufactures arrays of cDNA by applying polylysine to glass slides. Arrays of cDNA are printed onto the coated slides. The printed slides are then exposed to UV light to crosslink the DNA with the polylysine, thereby immobilizing the cDNA to the array.

### SUMMARY OF THE INVENTION

The invention provides a method for generating arrays with a variety of densities, in particular, high density arrays (e.g., an array having a density of about 10,000 to 100,000 spots per square centimeter or a pitch of between about 30 to about 100 micrometers).

Generally, the method includes a printing step and an illumination step. In the printing step, a volume (between about 0.5 picoliter and 500 picoliters) of a reagent solution containing receptor molecules is applied to a solid support in a desired pattern. In one embodiment, the receptor molecule is derivatized with a photoreactive agent. In an alternate embodiment, the solid support includes a photoreactive agent. Generally, the center to center distance of the pattern spots is between about 200 μm and 1 mm and the diameter of the spots is generally between about 100 μm and 500 μm. In a preferred embodiment, the receptor molecule is a nucleic acid (e.g., oligonucleotide, cDNA or PCR product).

In the illumination step, the photoreactive groups are irradiated to immobilize the receptor molecule to the solid support. In one embodiment, a mask having the same center to center distance (e.g., “pitch”) as the printed spots, but a smaller spot diameter, is placed over the printed pattern and illuminated. Preferably the mask illuminates spots having smaller diameters than the printed spots. Thus, according to the invention, the immobilized reagent spot has a smaller diameter than the original printed spot. In an alternate embodiment, the illumination step can be carried out using mirrored laser technology.

Typically, after the illumination step, reagent (e.g., receptor molecule) that has not been immobilized is removed by a wash step. The process can then be repeated, although offset from the original pattern. If desired, the process can be repeated multiple times to manufacture a high-density array.

### BRIEF DESCRIPTION OF THE FIGURES

FIG. 1 is a flow chart of the process of the invention.

FIGS. 2A and 2B are a schematic depiction of the process of the invention.

FIG. 3 is a schematic of an alternate process of the invention.

### DETAILED DESCRIPTION

The term “photolithography” refers to a process by which exposure of a surface to electromagnetic radiation in a defined pattern results in the generation of that pattern (or the negative of that pattern) on the surface. Typically, the pattern is generated by the formation or breaking of bonds. “Photolithography” can include masking techniques and other techniques, such as mirrored laser illumination.

As used herein, “reagent solution” refers to a solution that includes a receptor molecule. Typically, the reagent solution also includes a buffer. Generally, an array is prepared using at least one, more typically a plurality, of “reagent solutions”, each of which include a different receptor molecule such that an array is formed with different receptor molecules at distinct locations on the array.

As used herein, “receptor molecule” refers to a member of a binding pair that is to be immobilized onto the solid support. In a preferred embodiment, the receptor molecule is a nucleic acid. However, the receptor molecule can be any other molecule that specifically binds to a ligand. For example, the receptor molecule can be a protein, such as an immunoglobulin, a cell receptor, such as a lectin, or a fragment thereof (e.g., F_{ab} fragment, F_{ab′} fragments, etc . . . ).

As used herein, “target ligand,” or “target” refers to a ligand, such as a nucleic acid sequence, suspected to be present in a sample that is to be detected and/or quantitated in the method or system of the invention. In one embodiment, the nucleic acid comprises a gene or gene fragment to be detected in a sample. The term “sample” is used in its broadest sense. The term includes a specimen or culture suspected of containing target ligand.

As used herein, the terms “complementary” or “complementarity,” when used in reference to nucleic acids (i.e., a sequence of nucleotides such as an nucleic acid or a target nucleic acid), refer to sequences that are related by the base-pairing rules developed by Watson and Crick. For example, for the sequence “T-G-A” the complementary sequence is “A-C-T.” Complementarity may be “partial,” in which only some of the bases of the nucleic acids are matched according to the base pairing rules. Alternatively, there may be “complete” or “total” complementarity between the nucleic acids. The degree of complementarity between the nucleic acid strands has effects on the efficiency and strength of hybridization between the nucleic acid strands.

The terms “complementary,” or “complementarity,” when used in combination with molecules other than nucleic acids, refers to molecules that are capable of binding with a binding partner, such as molecules that are members of a specific binding pair.

The term “hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, the melting temperature (Tₘ) of the formed hybrid, and the G:C to A:T ratio within the nucleic acids.

As used herein, the term “nucleic acid” refers to any of the group of polynucleotide compounds having bases derived from purine and pyrimidine. The term “nucleic acid” may be used to refer individual nucleic acid bases or oligonucleotides (e.g., a short chain nucleic acid sequence of at least two nucleotides covalently linked together, typically less than about 500 nucleotides in length, and more typically between 20 to 100 nucleotides in length). The term “nucleic acid” can also refer to long sequences of nucleic acid, such as those found in cDNAs or PCR products (e.g., sequences of hundreds or thousands of nucleotides in length). The exact size of the nucleic acid sequence will depend upon many factors, which in turn depend upon the ultimate function or use of the nucleic acid.

Nucleic acids can be prepared using techniques presently available in the art, such as solid support nucleic acid synthesis, DNA replication, reverse transcription, etc. Alternately, nucleic acids can be isolated from natural sources. The nucleic acid can be in any suitable form, e.g., single stranded, double stranded, or as a nucleoprotein. A nucleic acid will generally contain phosphodiester bonds, although, in some cases, a nucleotide may have an analogous backbone, for example, a peptide nucleic acid (PNA). Nucleic acids include deoxyribonucleic acid (DNA) (such as complementary DNA (cDNA)), ribonucleic acid (RNA), and peptide nucleic acid (PNA). The nucleic acid may contain DNA, both genomic and cDNA, RNA or both, wherein the nucleic acid contains any combination of deoxyribo-and ribo nucleotides. Furthermore, the nucleic acid may include any combination of uracil, adenine, guanine, thymine, cytosine as well as other bases such as inosine, xanthenes, hypoxanthine and other non-standard or artificial bases.

PNA is a DNA mimic in which the native sugar phosphate DNA backbone has been replaced by a polypeptide. This substitution is said to increase the stability of the molecule, as well as improve both affinity and specificity.

Overview

Generally, the invention provides a method for generating a microarray. A microarray generally includes a solid support to which different receptor molecules are attached, each located in a predefined region physically separated from other regions.

While the invention will be described with particular reference to nucleic acids (and their ability to specifically “bind” via hybridization), it is understood that the invention has applicability to other specific binding agents as well, such as immunological binding pairs or other ligand/anti-ligand binding pairs or even proteins for which a ligand has yet to be found, such as targets for drug discovery.

Although the method is suitable for generating arrays with a variety of densities, the method is particularly well suited for generating high-density arrays. As used herein, the term “high density array” refers to a microarray having a density of more than 1,000 spots of receptor molecule per square centimeter, typically more than 5,000 spots per square centimeter, most typically between 10,000 and 100,000 spots per square centimeter. Generally, in a “high density array”, the spots are immobilized at a “pitch” between about 30 to about 100 micrometers (e.g., a distance from center to center between about 30 to about 100 micrometers). In contrast, most commercially available microarrays made by printing techniques have a density of approximately 100 to 1000 spots per square centimeter. Generally, in most commercially available arrays, the spots are immobilized at a pitch between about 100 to about 200 micrometers from center to center.

As used herein, a “spot” refers to a localized area that contains at least one, more typically a plurality, of a particular receptor molecule. Preferably, each “spot” contains a different receptor molecule. “Spot pattern” refers to the configuration of the spots on the surface of the solid support. In some instances, it may be desirable to have a uniform spot pattern, wherein each spot is separated from all neighboring spots by a predetermined distance. However, it is not necessary to have a uniform spot pattern (e.g., distance between one spots and all neighboring spots may not the same).

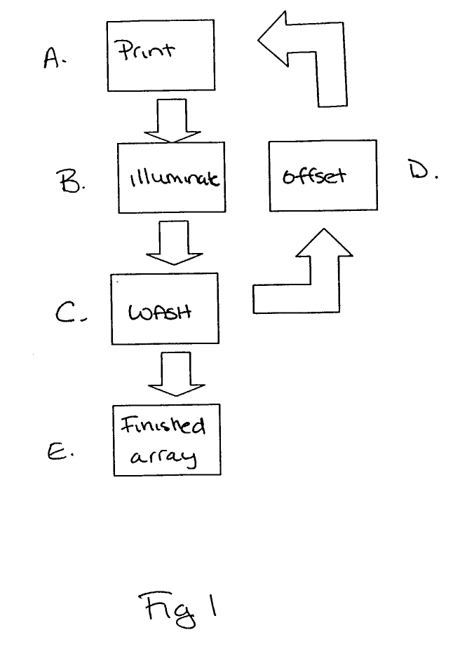

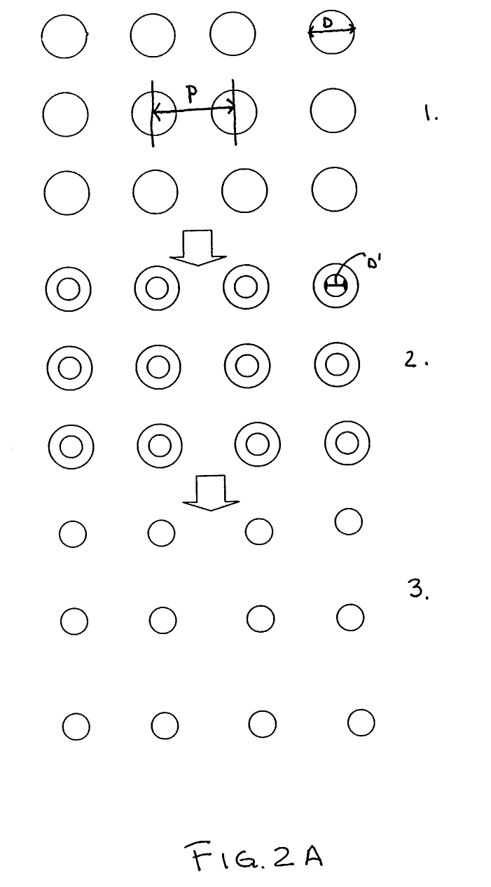

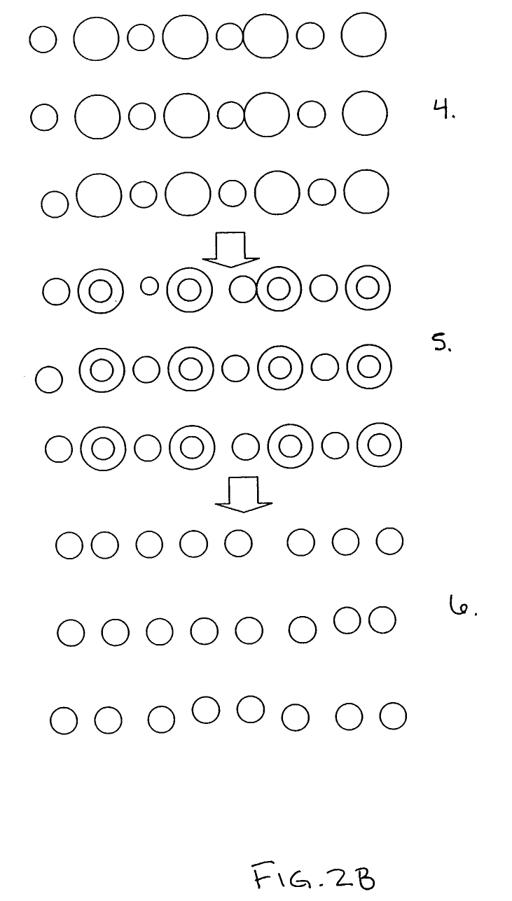

Generally, the method includes a printing step and an illumination step. The process is shown schematically in FIGS. 1 and 2. In the printing step (FIG. 1, step A and FIG. 2A, step 1), a predetermined volume (between about 0.5 picoliters and 500 picoliters) of a reagent solution is applied to a solid support in a desired pattern. Generally, the center to center distance of the printed spots (P) is between about 200 μm and 1000 μm and the diameter of the printed spots (D) is generally between about 100 μm and 500 μm.

In one embodiment, the receptor molecule is derivatized with at least one type of photoreactive group. As used herein, the term “type” refers to the reactive group. For example, one “type” of photoreactive group is an azide and another “type” of photoreactive group is an aryl ketone. Thus, a receptor molecule may be derivatized with multiple copies of one type of photoreactive group. Alternately, the receptor molecule may be derivatized with one or more copies of a variety of types of photoreactive groups. (The same concept applies to the following alternatives). In an alternate embodiment, the solid support contains at least one type of photoreactive group. Other alternatives are also envisioned, for example, both the receptor molecule and the solid support can include at least one type of photoreactive group. In another embodiment, the receptor molecule and solid support can include complementary elements of a photoreactive group, such that, upon illumination, the elements will interact to form a stable, preferably covalent, bond. In yet another embodiment, the reagent solution that is applied to the solid support prior to illumination can include at least one type of photoreactive group.

In the illumination step (shown in FIG. 1, step B and FIG. 2A, step 2) the photoreactive groups are irradiated such that a reaction is initiated that immobilizes the receptor molecule to the solid support. In one embodiment, a mask having the same center to center distance or “pitch” (P) as the printed spots is placed over the printed pattern and illuminated. As used herein, the term “same” means that the pitch of the spots is the same within the precision of the instrument used. Thus, there could be some slight variance between the center to center distances, but generally, the variance is negligible.

Preferably the mask permits radiation to illuminate the printed spots at a smaller diameter (D′) than the diameter (D) of the printed spot themselves, such that the spot of immobilized receptor molecule has a smaller diameter (D′) than the printed spot (D). Alternately, the illumination step can be accomplished using mirrored laser techniques.

Typically, after the illumination step, receptor molecule that has not been immobilized is removed by a wash step (FIG. 1, step C and FIG. 2A, step 3). The process can then be repeated, although offset from the existing spot pattern(s) (FIG. 1, step D and FIG. 2B). As used herein, the term “offset” refers to location of the immobilized spot. The printed spots may or may not overlap. The term “existing spot” refers to any immobilized spot pattern on the surface. If desired, the process can be repeated multiple times to manufacture a high-density array.

For example, if the printed spots have a diameter of 100 μm and a pitch of 200 μm (center to center), and the photoactivated spots have a diameter of 20 μm (with the same pitch as the printed spots), the mask can be offset to accommodate 25 arrays within the same space, resulting in a 25-fold increase in array density. Thus, if one has the ability make an array having 2500 spots per cm² by printing, using the method of the invention, an array having 62,500 spots per cm² can be prepared.

Advantageously, only one mask is needed for the method of the invention (although, more than one mask may be used if desired). If mirrored laser illumination is used, no masks are required. Thus, the method of the invention can provide a significant reduction in the cost of manufacture of high-density arrays as compared to photolithographic in situ solid phase synthesis, which requires multiple masks. Furthermore, longer nucleic acid sequences can be immobilized (including even cDNAs) than with in situ solid phase synthesis and the sequences can be purified prior to immobilization.

The number of spots per array may depend on the size and composition of the array, as well as the end use of the array. For certain diagnostic arrays, only a few different spots may be required; while other uses, such as expression analysis, may require more spots to collect the desired information.

Nucleic Acids

According to the method of the invention, a reagent solution containing receptor molecule is printed onto a solid support. The receptor molecule is preferably a nucleic acid, obtained from a natural source or synthesized using any suitable method. Methods for synthesizing nucleic acids are known. For example, nucleic acids may be prepared by conventional techniques such as polymerase chain reaction or biochemical synthesis, and then purified.

The length of the nucleic acid (i.e., the number of nucleotide bases) can vary widely, from 5 bases to several thousand bases. Preferably, the nucleic acid is at least 10 bases in length, to achieve specific hybridization. Nucleic acids with sequences ranging from about 10 to 500 bases are typical, as are sequences of about 20 to 200 bases, and those with 40 to 100 bases. Advantageously, the method of the invention can be used to generate arrays with longer nucleic acid sequences than are readily obtainable by photolithographic in situ solid phase synthesis of the nucleic acid sequence on the substrate surface. For example, nucleic acids of more than 30 bases can be used, as can nucleic acids of more than 40, more than 50 bases, or even more than 100 bases. That is, cDNAs and PCR products can be immobilized on the solid support using the method of the invention. Generally, nucleic acids having longer sequences (e.g., greater than 25 bases) are preferred, since higher stringency hybridization and wash conditions may be used, thereby decreasing or eliminating non-specific hybridization. However, shorter nucleic acids may be used if desired.

Substrate

According to the invention, the receptor molecules are immobilized on a solid support, also referred to herein as a substrate. Generally, the term “solid support” or “substrate” refers to a material that is insoluble in the solvent used and provides a two- or three- dimensional surface on which the nucleic acids can be immobilized. The composition of the solid support may be anything to which the receptor molecules may be attached, preferably covalently. The composition of the solid support may vary, depending on the method by which the receptor molecules are to be attached.

Preferably, the support surface does not interfere with receptor-ligand binding and is not subject to high amounts of non-specific binding. Suitable materials include biological or nonbiological, organic or inorganic materials. Suitable solid supports include, but are not limited to, those made of plastics, functionalized ceramic, resins, polysaccharides, functionalized silica, or silica-based materials, functionalized glass, functionalized metals, films, gels, membranes, nylon, natural fibers such as silk, wool and cotton and polymers. As used herein, the term “functionalized” refers to the addition of an organic modification to an inorganic surface, by known methods, to provide bonds with which the photoreactive groups can react. Polymeric surfaces are preferred, and suitable polymers include, but are not limited to polystyrene, polyethylene, polyethylene tetraphthalate, polyvinyl acetate, polyvinyl chloride, polyacrylonitrile, polymethyl methacrylate, butyl rubber, styrenebutadiene rubber, natural rubber, polypropylene, polyvinylidenefluoride, polycarbonate and polymethylpentene.

As mentioned above, the solid support can provide a two-dimensional surface or a three-dimensional surface. A three-dimensional surface can be provided using a solid support of a desired length, width and thickness that is permeable to allow the nucleic acids to migrate into the pores or matrix. Because the nucleic acids can be immobilized along the length, width and height (thickness) of the solid support, a higher density of nucleic acids can be immobilized in a given area on a three-dimensional surface than on a two-dimensional surface.

Preferably a surface is selected that will reduce non-specific adsorption of the nucleic acids to the solid support. Generally, a hydrophilic surface will reduce non-specific adsorption.

“Hydrophilic” and “hydrophobic” are used herein to describe compositions broadly as water loving and water hating, respectively. Generally, hydrophilic compounds are relatively polar and often ionizable. Such compounds usually bind water molecules strongly. Hydrophobic compounds are usually relatively non-polar and non-ionizing. Hydrophobic surfaces will generally cause water molecules to structure in an ice-like conformation at or near the surface. Hydrophobic and hydrophilic are relative terms and are used herein in the sense that various compositions, liquids and surfaces may be hydrophobic or hydrophilic relative to one another.

The dimensions of the solid support can vary and may be determined by such factors as the dimensions of the desired array, and the amount of diversity desired. In one embodiment, the nucleic acids are immobilized on a substrate in the form of a sheet or film that is subsequently cut into individual arrays. Alternately, individual arrays can be manufactured independently. The solid supports may also be singly or multiply positioned on other supports, such as microscope slides.

As indicated, in some embodiments, photoactivatable nucleic acids (i.e., receptor molecules derivitized with a photogroup), are immobilized on surfaces. The photoactivatable nucleic acids of the invention can be applied to any surface having carbon-hydrogen bonds with which the photoactivatable groups can react to immobilize the nucleic acids to surfaces. Examples of appropriate substrates include, but are not limited to, polypropylene, polystyrene, poly(vinyl chloride), polycarbonate, poly(methyl methacrylate), parylene and any of the numerous organosilanes used to pretreat glass or other inorganic surfaces. The photoactivatable nucleic acids can be printed onto surfaces in arrays, then photoactivated by uniform illumination to immobilize them to the surface in specific patterns. They can also be sequentially applied uniformly to the surface, then photoactivated by illumination through a series of masks to immobilize specific sequences in specific regions. Thus, multiple sequential applications of specific photoderivatized nucleic acids with multiple illuminations through different masks and careful washing to remove uncoupled photo-nucleic acids after each photocoupling step can be used to prepare arrays of immobilized nucleic acids. The photoactivatable nucleic acids can also be uniformly immobilized onto surfaces by application and photoimmobilization.

Printing

According to the invention, a volume of a reagent solution containing receptor is applied to a solid support at a selected position. The reagent solution may be applied to the substrate using known techniques, for example, using a modified commercially available printing instrument. For example, a commercially available printing instrument may need to be modified to allow for the illumination processes of the invention. Preferably, an automated x-y-z positioner is used for accurate and repeated spotting of reagent onto the solid support. Preferably, the x-y-z positioner has an accuracy of at least 10 μm in all three (x, y and z) directions. Generally, spotting robots do not require sensors or visual referencing.

Generally, in the printing stage, a small volume (e.g., between 0.1 picoliters and 1 nanoliter, more typically between 0.5 picoliters and 500 picoliters) of a reagent solution containing the desired receptor molecule is applied to the substrate surface. The diameter of the printed spots may vary, depending on the substrate surface and the volume and viscosity of the solution applied. Typically, the printed spots have a diameter (D) between about 100 to 500 μm. The pitch (P) is generally influenced by the diameter of the spots. Generally, the pitch (P) is two or more times the diameter of the spots (e.g., the pitch is generally between 200 μm and 1000 μm).

Photoreactive Groups on the Substrate Surface

In one embodiment, the solid support includes a surface coated with at least one type of photoreactive group.

Photoreactive groups are defined herein, and preferred groups are sufficiently stable to be stored under conditions in which they retain such properties. See, e.g., U.S. Pat. No. 5,002,582, the disclosure of which is incorporated herein by reference. Latent reactive groups can be chosen that are responsive to various portions of the electromagnetic spectrum, with those responsive to ultraviolet and visible portions of the spectrum (referred to herein as “photoreactive”) being particularly preferred.

Photoreactive groups respond to specific applied external stimuli to undergo active specie generation with resultant covalent bonding to an adjacent chemical structure, e.g., as provided by the same or a different molecule. Photoreactive groups are those groups of atoms in a molecule that retain their covalent bonds unchanged under conditions of storage but that, upon activation by an external energy source, form covalent bonds with other molecules.

As used herein, “photoreactive groups” include at least one reactive moiety that responds to a specific applied external energy source, such as radiation, to undergo active species generation (e.g., free radicals such as nitrenes, carbenes and excited ketone states) with resultant covalent bonding to an adjacent chemical structure. Photoreactive groups may be chosen to be responsive to various portions of the electromagnetic spectrum, typically ultraviolet, visible or infrared portions of the spectrum. “Irradiation” refers to the application of electromagnetic radiation to a surface.

According to one embodiment, the receptor molecule to be immobilized on the surface may or may not be modified with a photoreactive group.

For example, the solid support may include a glass substrate having a polycationic polymer coating. In this embodiment, the polymer coating includes a cationic polypeptide, such as polylysine or polyarginine. Such a solid support may be prepared using known techniques. For example, the slide may be prepared by placing a uniform-thickness film of the polycationic polymer on the surface of the slide to form a film that is then dried to form the coating. The amount of a polycationic polymer added is preferably sufficient to form at least a monolayer of polymers on the solid support surface. The film is generally bound to the surface via electrostatic binding between negative silyl-OH groups on the surface and charged amine groups in the polymers. Poly-l-lysine coated glass slides are also commercially available, for example, from Sigma Chemical Co. (St. Louis Mo.). Nucleic acid sequences can be printed on such a surface and then illuminated to cross-link the nucleic acids to the cationic polymer.

Photoreactive Groups Attached to the Receptor Molecule

In an alternate embodiment, the receptor molecules are derivatized with one or more of at least one type of photoreactive group that can be activated to immobilize the receptor molecule to the support surface. According to this embodiment, the photo-derivatized receptor molecule is covalently immobilized to the support surface by the application of suitable irradiation. The photoreactive groups are preferably covalently bound, directly or indirectly, at one or more points along the receptor molecule. One or more photogroups can be bound to the receptor molecule in any suitable fashion. For example, if the receptor molecule is a nucleic acid, the nucleic acid may be synthesized with at least one derivatized nucleic acid base. Alternately, a naturally occurring or previously synthesized nucleic acid can be derivatized in such a manner as to provide a photogroup at the 3′ terminus, at the 5′-terminus, along the length of the nucleic acid itself, or any combination thereof.

The oligonucleotide component of a photoactivatable oligonucleotide can be synthesized using any suitable approach, including methods based on the phosphodiester chemistry and more recently, on solid-phase phosphoramidite techniques. See generally T. Brown and D. Brown, “Modern Machine-Aided Methods of Oligonucleotide Synthesis”, Chapter 1, pp. 1-24 in Oligonucleotides and Analogues, A Practical Approach, F. Eckstein, ed., IRL Press (1991), the disclosure of which is incorporated herein by reference.

The stepwise synthesis of oligonucleotides generally involves the formation of successive diester bonds between 5′-hydroxyl groups of bound nucleotide derivatives and the 3′-hydroxyl groups of a succession of free nucleotide derivatives. The synthetic process typically begins with the attachment of a nucleotide derivative at its 3′-terminus by means of a linker arm to a solid support, such as silica gel or beads of borosilicate glass packed in a column. The ability to activate one group on the free nucleotide derivative requires that other potentially active groups elsewhere in the reaction mixture be “protected” by reversible chemical modifications. The reactive nucleotide derivative is a free monomer in which the 3′-phosphate group has been substituted, e.g., by dialkylphosphoramidite, which upon activation reacts with the free 5′-hydroxyl group of the bound nucleotide to yield a phosphite triester. The phosphite triester is then oxidized to a stable phosphotriester before the next synthesis step.

The 3′-hydroxyl of the immobilized reactant is protected by virtue of its attachment to the support and the 5′-hydroxyl of the free monomer can be protected by a dimethoxytrityl (DMT) group in order to prevent self-polymerization. A 2-cyanoethyl group is usually used to protect the hydroxyl of the 3′-phosphate. Additionally, the reactive groups on the individual bases are also protected. A variety of chemistries have been developed for the protection of the nucleotide exocyclic amino groups. The use of N-acetyl protecting groups to prepare N-acetylated deoxynucleotides has found wide acceptance for such purposes.

After each reaction, excess reagents are washed off the columns, any unreacted 5′-hydroxyl groups are blocked or “capped” using acetic anhydride, and the 5′-DMT group is removed using dichlorolacetic acid to allow the extended bound oligomer to react with another activated monomer in the next round of synthesis.

Finally, the fully assembled oligonucleotide is cleaved from the solid support and deprotected, to be purified by HPLC or some other method. The useful reagents and conditions for cleavage depend on the nature of the linkage. With ester linkages, as are commonly provided by linkage via succinyl groups, cleavage can occur at the same time as deprotection of the bases using concentrated aqueous ammonium hydroxide.

A review of methods for modifying nucleic acids is contained in (Bioconjugate Chem., 3(1):165-186, 1990). Methods described include modifications introduced during oligonucleotide synthesis, enzymatic modifications, and chemical modifications of native or post synthetic DNA. Reagents could be designed to incorporate photogroups onto nucleic acids using all of these strategies.

Numerous different reagent types could be designed to incorporate photogroups during synthesis using the phosphoramidite method. One type of photoreagent has two reactive groups which can be differentially protected. An example is a reagent containing a photogroup and side-chain(s) with a primary and a secondary alcohol. The primary alcohol is protected with a DMT group. This reagent could be used to provide a photogroup at the 3′-end of the DNA by creating an ester link between the secondary alcohol and a silica support containing carboxylic acid groups. In order to put photogroups at any other site during the synthesis, the secondary alcohol is reacted with an appropriately protected chlorophosphoramidite (i.e. 2-cyanoethyl diisopropylchlorophosphoramidite). This reagent is used in the same manner as protected nucleotides are currently used for DNA synthesis. A reagent having a photogroup and just one hydroxyl could be derivatized with a chlorophosphoramidite to create a 5′-end derivatization reagent. In a similar manner, reagents could be designed to provide photogroups during oligonucleotide synthesis using chemistry other than the phosphoramidite method.

Post-synthetic derivatization of the oligonucleotides is also possible. One way to accomplish this is to incorporate an amine group into the oligonucleotide during synthesis. Reagents are commercially available to incorporate an amine at the 5′-end of the oligonucleotide. Various chemical approaches could be used to add a photogroup to the amine derivatized DNA. One example is to use a reagent containing a photogroup and an N-oxysuccinimide ester (NOS). The NOS ester is reacted with the amine, thereby incorporating the photogroup.

Nucleic acids could be prepared having the photoreactive groups along the backbone of the molecule as opposed to having the groups at either the 3′- or 5′-end. A number of approaches can be envisioned for the preparation of such a photo-nucleic acid reagent. For example, the bases present on the nucleotides making up the nucleic acid possess numerous reactive groups which could be photoderivatized using a heterobifunctional photoreagent possessing a photogroup and a chemically reactive group suitable for covalent coupling to the bases. This process would result in a relatively nonselective derivatization of the nucleic acid in terms of the location along the backbone as well as the number of photogroups.

In a further example, the nucleotide building blocks typically used in DNA synthesis could be derivatized with a photoreactive group by attachment of the photogroup to one of the reactive functionalities present on the base residue of the nucleotide. Use of the resulting reagent in an automated synthesizer with typical reaction conditions would permit incorporation of the photogroup at designated points along the chain of the oligo. In a preferred example, there are numerous commercial non-nucleotide reagents that are used to introduce reactive groups such as amines in specific locations along the backbone of the oligo during a typical DNA synthesis. Following completion of the oligo synthesis incorporating these reactive groups, the photoreactive group would then be introduced by reaction with these reactive sites. Alternatively, these non-nucleotide reagents could be photoderivatized prior to their use in the oligo synthesis.

The photoreactive group provides a derivatized receptor molecule that can be selectively and specifically activated in order to attach the receptor molecule to a support in a manner that substantially retains chemical and/or biological function. According to this embodiment, “direct” attachment of the photoreactive group means that the photoreactive compound is attached directly to the receptor molecule. On the other hand, “indirect” attachment refers to attachment of a photoreactive compound and receptor molecule to a common structure, such as a synthetic or natural polymer. The resulting photo-derivatized receptor molecule can be covalently immobilized by the application of suitable irradiation, and usually without the need for surface pretreatment, to a variety of substrate surfaces. The method of this embodiment involves both the thermochemical attachment of one or more photoreactive groups to a receptor molecule and the photochemical immobilization of that receptor molecule derivative upon a substrate surface.

In the method of “indirect” attachment oligos could be incorporated in reagents of the invention by attaching the intact oligo as a ligand along the backbone of a polymer. A number of approaches can be envisioned for the preparation of such a polymeric photo-oligo reagent. In one example, the oligo could be prepared in monomer form by covalent attachment of a polymerizable vinyl group such as acryloyl to the oligo, either at the ends or along the backbone. This could be accomplished by reaction of acryloyl chloride with an amine derivatized oligo. These oligo monomers could then be copolymerized with a photoderivatized monomer along with other comonomers such as acrylamide or vinylpyrrolidone. The resulting polymer would have the photogroups and oligos randomly attached along the backbone of the polymer. Alternatively, the polymer could be prepared with the photoreactive group at one end of the polymer by use of a chain transfer reagent having a photogroup as part of the structure.

In a further extension of this approach, a preformed polymer could be derivatized with oligos in a second step. In this approach, a polymer is prepared having chemically reactive groups located, along the backbone of the polymer, each of which is capable of reacting with appropriately substituted oligos. For example, polymers possessing activated groups such as NOS esters could be reacted with oligos containing amine functionality resulting in covalent attachment of the oligo to the polymer backbone through an amide bond. This polymer could be prepared using a photoderivatized monomer or the photogroup could be added to the preformed polymer in a manner similar to the oligo. Alternatively, the polymer could be prepared with the photoreactive group at one end of the polymer by use of a chain transfer reagent having a photogroup as part of the structure. The oligo would then be added to the reactive groups in a second step.

The receptor molecule can be applied to any solid support, preferably those having carbon-hydrogen bonds with which the photoreactive groups can react to immobilize the nucleic acids to surfaces. Examples of appropriate substrates include, but are not limited to, polypropylene, polystyrene, poly(vinyl chloride), polycarbonate, poly(methyl methacrylate), parylene and any of the numerous organosilanes used to pretreat glass or other inorganic surfaces.

Preparation of a high density array using photo-derived receptor molecules is generally preferred over a method using photoreactive groups on the surface of the solid support because a surface that reduces non-specific adsorption of the nucleic acids (or other components) can be used.

Photo-derivatized nucleic acids, and methods for making the same are disclosed in detail in commonly assigned U.S. Patent application Ser. No. 09/028,806, entitled PHOTOACTIVATABLE NUCLEIC ACID DERIVATIVES. This application is commonly owned by the assignee of the present application, and the entire disclosure is incorporated herein by reference.

Photoreactive Groups

According to one embodiment, the receptor molecules are derivitized with photoreactive groups. Photoreactive aryl ketones are preferred, such as acetophenone, benzophenone, anthraquinone, anthrone, and anthrone-like heterocycles (i.e., heterocyclic analogs of anthrone such as those having N, O, or S in the 10-position), or their substituted (e.g., ring substituted) derivatives. Examples of preferred aryl ketones include heterocyclic derivatives of anthrone, including acridone, xanthone and thioxanthone, and their ring substituted derivatives. Particularly preferred are thioxanthone, and its derivatives, having excitation wavelengths greater than about 360 nm.

The azides are also a suitable class of photoreactive groups and include arylazides (C₆R₅N₃) such as phenyl azide and particularly 4-fluoro-3-nitrophenyl azide, acyl azides (—CO—N₃) such as ethyl azidoformate, phenyl azidoformate, sulfonyl azides (—SO₂—N₃) such as benzensulfonyl azide, and phosphoryl azides (RO)₂PON₃ such as diphenyl phosphoryl azide and diethyl phosphoryl azide. Diazo compounds constitute another class of photoreactive groups and include diazoalkanes (—CHN₂) such as diazomethane and diphenyldiazomethane, diazoketones (—CO—CHN₂) such as diazoacetophenone and 1-trifluoromethyl-1-diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as 3-trifluoromethyl-3-phenyldiazirine, and ketenes (—CH═C═O) such as ketene and diphenylketene.

Upon activation of the photoreactive groups, the receptor molecules are covalently bound to each other and/or to the material surface by covalent bonds through residues of the photoreactive groups. Exemplary photoreactive groups, and their residues upon activation, are shown as follows.

| Photoreactive | Group | Residue Functionality |
|---|---|---|
| aryl azides | amine | R—NH—R′ |
| acyl azides | amide | R—CO—NH—R′ |
| azidoformates | carbamate | R—O—CO—NH—R′ |
| sulfonyl azides | sulfonamide | R—SO-₂-NH—R′ |
| phosphoryl azides | phosphoramide | (RO)₂PO—NH—R′ |
| diazoalkanes | new C—C bond | |
| diazoketones | new C—C bond and ketone | |
| diazoacetates | new C—C bond and ester | |
| beta-keto-alpha- | new C—C bond and beta- | |
| diazoacetates | ketoester | |
| aliphatic azo | new C—C bond | |
| diazirines | new C—C bond | |
| ketenes | new C—C bond | |
| photoactivated | new C—C bond and alcohol | |
| ketones | | |

The photoactivatable nucleic acids can be printed onto surfaces in arrays, then photoactivated by uniform illumination to immobilize them to the surface in specific patterns. They can also be sequentially applied uniformly to the surface, then photoactivated by illumination through a series of masks to immobilize specific sequences in specific regions. Thus, multiple sequential applications of specific photoderivatized nucleic acids with multiple illuminations through different masks and careful washing to remove uncoupled photo-nucleic acids after each photocoupling step can be used to prepare arrays of immobilized nucleic acids. The photoactivatable nucleic acids can also be uniformly immobilized onto surfaces by application and photoimmobilization.

Illumination

According to the invention, after the reagent solution is printed onto the solid support, at least some of the receptor molecules are immobilized onto the solid support in an illumination step.

In one embodiment, as discussed above, the illumination step is used to immobilize the nucleic acids in an essentially circular configuration having a diameter that is less than the diameter of the printed spot. As used herein, the term “essentially circular” means that the shape is generally that of a circle, although some irregularities may be present. For example, the shape may be slightly oval or the edge defining the shape may not be completely smooth. Additionally, the illumination step can be used to generate a “spot” of immobilized nucleic acids having a non-circular configuration. For example, the nucleic acids can be immobilized in the shape of a square, triangle, cross, dash, etc. Specially shaped “spots” could facilitate detection of hybridization patterns. Generally, the area defined by the illuminated spot is less than the area defined by the diameter of the printed spot. The area (A) defined by the diameter (D) of the essentially circular printed spot refers to the area calculated by the formula: Area=π(D/2)².

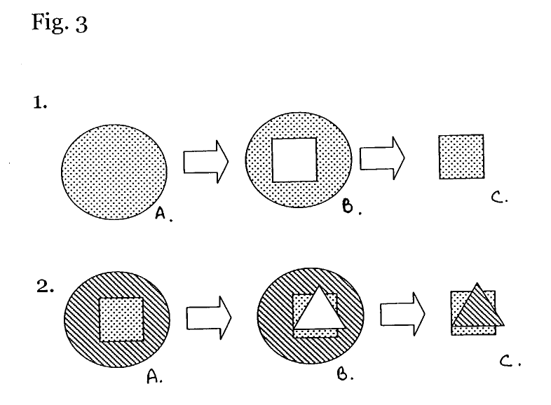

In another embodiment, “spots” of differing shapes could be superimposed over one another. (FIG. 3) For example, a first nucleic acid sequence could be printed onto the solid support (FIG. 3(1)(A)) and illuminated with a square shaped light pattern (such that the nucleic acids are immobilized in a square; FIG. 3(1)(B)). Non-immobilized nucleic acid is removed (FIG. 3(1)(C)) before a second nucleic acid is printed onto the solid support (FIG. 3(2)(A)). This time, the nucleic acid might be illuminated with a triangular light pattern (FIG. 3(2)(B)). Again, excess nucleic acid is removed. Using this technology, an array can be prepared wherein a square shaped spot will be detected in the presence of one type of target ligand and a triangular shaped spot will be detected in the presence of a different ligand. In another embodiment, the spots having differing configurations can be offset.

Masked Illumination

In one embodiment, the receptor molecules are immobilized to the solid support by masked illumination. As used herein, the term “immobilized” means the receptor molecule is stably attached to the support surface. Such attachment is preferably covalent, although other suitable stable attachment is also contemplated.

Generally, techniques for using masks to control radiation directed immobilization of the receptor molecule to a solid support are known. Briefly, the present invention used a mask (e.g., a chrome or glass mask) to direct the immobilization of receptor molecule onto the solid support. According to the invention, the printed spots are illuminated through a mask having openings at the same pitch (center to center distance) as the printed spots. However, the diameter of illumination at each printed spot is preferably less than the diameter of the printed spot itself. Thus, the diameter of the immobilized receptor molecule is less than the diameter of the printed spot.

Preferably, the mask has a pitch from between about 100 μm to about 500 μm from center to center, more preferably between about 100 μm to about 200 μm. Preferably, the illumination diameter for each spot is less than 100 82 m, preferably less than 50 μm. The illumination diameter can be between about 10 μm and 50 μm, more typically between 20 μm and 40 μm. In some cases it may be desirable to have an illumination diameter of less than 10 μm. A limiting factor may be wavelength of light used and/or the resolution of the detection system.

The wavelength may be determined, at least in part, by the photoreactive groups used to immobilize the receptor molecule. That is, a given photoreactive groups are preferably illuminated with light of a particular wavelength.

Mirrored Laser Illumination

As an alternative to photolithography, mirrored laser illumination may be used to immobilize the receptor molecules to the solid support. According to this embodiment, a digital micromirror is used to direct radiation onto specific areas of the printed spots to immobilize the receptor molecule on the solid support. For example, a suitable digital micromirror array may be Texas Instrument's (Dallas, Tex.) Digital Micromirror Device (DMD) commonly used in computer display projection systems. The mirrors can be individually positioned and can be used to create any given pattern or image in a broad range of wavelengths.

An advantage of mirrored laser illumination includes the lower cost when compared to photolithographic in situ solid phase synthesis of the nucleic acids (e.g., adjusting the mirrors in the micromirror device is cheaper than creating multiple masks).

Methods of Use

The microarray of the invention may be used for high throughput (large scale hybridization assays) and cost-effective analysis of complex mixtures. For example, the assay is suitable for genetic applications, including but not limited to, DNA sequencing, genetic diagnosis, and genotyping of organisms.

The arrays can be adapted to detect a wide variety of nucleic acids in a biological sample. In use, the array can be exposed to a sample suspected of containing one or more target ligands, under conditions suitable to permit the target ligands to hybridize to their corresponding complement on the array. The presence or absence of the target nucleic acid on the assay array can be determined with a chosen signal generation and detection system. Such detection methods are known in the art.

For gene mapping, a gene or a cloned DNA fragment is hybridized to an ordered array of DNA sequences, and the identity of the DNA elements applied to the array is established by the pattern detected on the array. In constructing physical maps of the genome, arrays of immobilized cloned DNA fragments are hybridized with other cloned DNA fragments to establish whether the cloned fragments in the probe mixture overlap and are therefore contiguous to the immobilized clones on the array.

The arrays of immobilized DNA sequences may also be used for genetic diagnostics. For example, an array containing multiple forms of a mutated gene or genes can be probed with a labeled mixture of a patient's DNA that will preferentially interact with only one of the immobilized versions of the gene.

Arrays of immobilized DNA sequences can also be used in DNA probe diagnostics. For example, the identity of a pathogenic microorganism can be established by hybridizing a sample of the unknown pathogen's DNA to an array containing many types of known pathogenic DNA. A similar technique can also be used for genotyping of an organism. Other molecules of genetic interest, such as cDNA's and RNAs can be immobilized on the array or alternatively used as the labeled probe that is applied to the array.

In one embodiment, target nucleic acids (referred to herein as a “ligand”) may be labeled with a detectable label. The label may be incorporated at a 5′ terminal site, a 3′ terminal site, or at an internal site within the length of the nucleic acid. Alternately, a “sandwich” assay can be used. In a sandwich assay, a capture probe is immobilized on the substrate surface and is contacted with a target ligand to form an attachment complex. The capture probe is designed such that it binds to a particular sub-part of the ligand. The attachment complex is then contacted with a labeled detection probe that binds to another sub-part of the ligand. Preferred detectable labels include a radioisotope, a stable isotope, an enzyme (typically used in combination with a chromogenic substrate), a fluorescent chemical, a luminescent chemical, or a chromatic chemical. There are many known procedures for incorporating a detectable label into a nucleic acid.

The invention has thus been described. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments.

### EXAMPLES

### Example 1

### Preparation and Evaluation of a Benzophenone Substituted Oligonucleotide

(a) Preparation of N-Succinimidyl 6-(4-Benzoylbenzamido)hexanoate (BBA-EAC-NOS)

4-Benzoylbenzoyl chloride, 60.00 g (0.246 moles), prepared as described in Example 3(a), was dissolved in 900 ml of chloroform. The 6-aminohexanoic acid, 33.8 g (0.258 moles), was dissolved in 750 ml of 1 N NaOH and the acid chloride solution was added with stirring. The mixture was stirred vigorously to generate an emulsion for 45 minutes at room temperature. The product was then acidified with 75 ml of 12 N HCl and extracted with 3×500 ml of chloroform. The combined extracts were dried over sodium sulfate, filtered, and evaporated under reduced pressure. The 6-(4-benzoylbenzamido)hexanoic acid was recrystallized from toluene/ethyl acetate (3/1) to give 77.19 g (93% yield) of product, m.p. 106.5-109.5° C.

The 6-(4-benzoylbenzamido)hexanoic acid, 60.00 g (0.177 mmoles), was added to a dry flask and dissolved in 1200 ml of dry 1,4-dioxane. N-Hydroxysuccinimide, 21.4 g (0.186 moles) was added followed by 41.9 g (0.203 moles) of 1,3-dicyclohexylcarbodiimide and the mixture was stirred overnight at room temperature under a drying tube to protect the reaction from moisture. After filtration to remove the 1,3-dicyclohexylurea, the solvent was removed under reduced pressure and the resulting oil was diluted with 300 ml of dioxane. Any remaining solids which formed were removed by filtration and after removal of solvent, the BBA-EAC-NOS was recrystallized twice from ethanol to give 60.31 g of a white solid, m.p. 123-126° C.

(b) Photoderivatization of an Amino-Modified Oligonucleotide

The 30-base oligomer (-mer) probe 5′-TTCTGTGTCTCCCGCTCCCAATACTCGGGC-3′ (ID1), synthesized with a 5′-amino-modifier containing a C-12 spacer (amine-ID1), was custom made at Midland Certified Reagent Company (Midland, Tex.). Oligonucleotide amine-ID1, 100 μg (10 nmole, 39.4 μl of 2.54 mg/ml stock in water) was mixed on a shaker in a microcentrifuge tube with 43.8 μg (100 nmole, 8.8 μl of 5 mg/ml stock in DMF) of BBA-EAC-NOS, prepared as described above in Example 1(a), and 4 μl of 1 M sodium bicarbonate buffer, pH 9. The reaction proceeded at room temperature for 3 hours. To remove unreacted BBA-EAC-NOS, the reaction was diluted with 148 μl phosphate buffered saline (PBS, 10 mM Na₂HPO₄, 150 mM NaCl, pH 7.2) and then loaded onto a NAP-5 column (Pharmacia Biotech, Uppsala, Sweden) according to the manufacturer's specifications. PBS was used to equilibrate the column and to elute the oligonucleotides off the column. The NAP-5 column, which contains Sephadex G-25 gel, separated oligonucleotides from the small molecular weight compound. A total of 3.1 A₂₆₀ units or 96 μg of benzophenone derivatized oligonucleotide ID1 was recovered.

(c) Evaluation of the Benzophenone Substituted Oligonucleotide

Oligos amine-ID1 and benzophenone-ID1 at 5 pmole/0.1 ml per well was incubated in polypropylene (PP, Corning Costar, Cambridge, Mass.) microwell plates in the incubation buffer (50 mM phosphate buffer, pH 8.5, 1 mM EDTA, 15% Na₂SO₄) at room temperature overnight. Half of the plates were illuminated with a Dymax lamp (Model no. PC-2, Dymax Corporation, Torrington, Conn.) which contained a Heraeus bulb (W. C. Heraeus GmbH, Hanau, Federal Republic of Germany) and a cutoff filter that blocked out all light below 300 nm. The illumination duration was for 2 minutes at an intensity of 1-2 mW/cm² in the wavelength range of 330-340 nm. The remaining half of the plates that were not illuminated served as the adsorbed oligo controls. All of the plates were then washed with PBS containing 0.05% Tween 20 using a Microplate Auto Washer (Model EL 403H, Bio-Tek Instruments, Winooski, Vt.).

Hybridization was performed as described below using the complementary 5′-Biotin-CGGTGGATGGAGCAGGAGGGGCCCGAGTATTGGGAGCGGGAGACACAGAA -3′ (ID2) detection probe or the non-complementary 5′-Biotin-CCGTGCACGCTGCTCCTGCTGTTGGCGGCCGCCCTGGCTCCGACTC AGAC -3′ (ID3) oligonucleotide. Both oligos were procured from the Mayo Clinic (Rochester, Minn.). The plates were blocked at 55° C. for 30 minutes with hybridization buffer consisting of 5×SSC (0.75 M NaCl, 0.075 M citrate, pH 7.0), 0.1% lauroylsarcosine, 1% casein, and 0.02% sodium dodecyl sulfate (SDS). When the detection probe was hybridized to the immobilized probe, an aliquot of 50 fmole of detection probe in 0.1 ml was added per well and incubated for 1 hour at 55° C. The plates were then washed with 2×SSC containing 0.1% SDS for 5 minutes at 55° C. The bound detection probe was assayed by adding 0.1 ml of a conjugate of streptavidin and horseradish peroxidase (SA-HRP, Pierce, Rockford, Ill.) at 0.5 μg/ml which was incubated for 30 minutes at 37° C. The plates were then washed with PBS/Tween, followed by the addition of peroxidase substrate (H₂O₂ and tetramethylbenzidine, Kirkegard and Perry Laboratories, Gaithersburg, Md.) and measurement at 655 nm, 20 minutes later, on a microwell plate reader (Model 3550, Bio-Rad Labs, Cambridge, Mass.).

The results listed in Table 1 show that the illuminated benzophenone-derivatized oligonucleotide provided a higher hybridization signal than the adsorbed oligonucleotide control. Conversely, there was no difference between the hybridization signals generated by the illuminated and the adsorbed non-derivatized oligonucleotides.

TABLE 1
Hybridization signals (A₆₅₅ ± standard
deviation) from amine-ID1 and benzophenone-ID1
on PP microwell plates.

| | |
|---|---|
| Adsorbed Control | Illuminated |

| | | Non- | | Non- |
|---|---|---|---|---|
| | Complem. | complem. | Complem. | complem. |
| | Det. | Det. | Det. | Det. |
| | ID2 | ID3 | ID2 | ID3 |
| Amine-ID1 | 0.289 ± | 0.014 ± | 0.250 ± | 0.069 ± |
| | 0.025 | 0.005 | 0.023 | 0.005 |
| Benzophenone- | 0.143 ± | 0.008 ± | 0.456 ± | 0.026 ± |
| ID1 | 0.034 | 0.007 | 0.027 | 0.005 |

### Example 2

### Preparation and Evaluation of a Psoralen Substituted Oligonucleotide

A 30-mer 5′-psoralen-ID1 was custom synthesized using psoralen-C₆-phosphoramidite by Midland Certified Reagent Company. A coating solution containing 1 mg/ml of a photoreactive polyvinylpyrrolidone (PV05, SurModics, Eden Prairie, Minn.) was prepared in water. Polypropylene microwells containing 0.1 ml of the PV05 coating solution were incubated at room temperature for 30 minutes. The solution was aspirated from the wells and the plates were illuminated for 2 minutes using the conditions described in Example 1(c) except no filter was used.

Oligos amine-ID1 and psoralen-ID1, at 5 pmole/0.1 ml per well were incubated in untreated and PV05-treated PP microwell plates in incubation buffer at room temperature overnight. The plates were illuminated and hybridized as described in Example 1(c). The results in Table 2 show that the illuminated psoralen derivatized oligonucleotide on PV05 treated PP surfaces had higher hybridization signals than the adsorbed control. Conversely, there was no difference between the hybridization signals generated by the illuminated and the adsorbed non-derivatized oligonucleotides.

TABLE 2
Hybridization signals (A₆₅₅ ± standard
deviation) from amine-ID1 and psoralen-ID1
on treated PP microwell plates.

| | |
|---|---|
| Adsorbed Control | Illuminated |

| | | Non- | | Non- |
|---|---|---|---|---|
| | Complem. | complem. | Complem. | complem. |
| | Det. | Det. | Det. | Det. |
| | ID2 | ID3 | ID2 | ID3 |
| Amine-ID1 | 0.210 ± | 0.016 ± | 0.351 ± | 0.094 ± |
| | 0.029 | 0.013 | 0.007 | 0.006 |
| Psoralen-ID1 | 0.094 ± | 0.022 ± | 0.554 ± | 0.056 ± |
| | 0.013 | 0.009 | 0.084 | 0.034 |

### Example 3

### Preparation and Evaluation of a Photopolymer Derivatized with Oligonucleotides

(a) Preparation of 4-Benzoylbenzoyl Chloride (BBA-C1)

4-Benzoylbenzoic acid (BBA), 1.0 kg (4.42 moles), was added to a dry 5 liter Morton flask equipped with reflux condenser and overhead stirrer, followed by the addition of 645 ml (8.84 moles) of thionyl chloride and 725 ml of toluene. Dimethylformamide, 3.5 ml, was then added and the mixture was heated at reflux for 4 hours. After cooling, the solvents were removed under reduced pressure and the residual thionyl chloride was removed by three evaporations using 3×500 ml of toluene. The product was recrystallized from toluene/hexane (1/4) to give 988 g (91% yield) after drying in a vacuum oven. Product melting point was 92-94° C. Nuclear magnetic resonance (NMR) analysis at 80 MHz (¹H NMR (CDCl₃)) was consistent with the desired product: aromatic protons 7.20-8.25 (m, 9H). All chemical shift values are in ppm downfield from a tetramethylsilane internal standard. The final compound was stored for use in the preparation of a monomer used in the synthesis of photoactivatable polymers as described, for instance, in Example 3(c) or for heterobifunctional compounds as described, for instance, in Example 1(a).

(b) Preparation of N-(3-Aminopropyl)methacrylamide Hydrochloride (APMA)

A solution of 1,3-diaminopropane, 1910 g (25.77 moles), in 1000 ml of CH₂Cl₂ was added to a 12 liter Morton flask and cooled on an ice bath. A solution of t-butyl phenyl carbonate, 1000 g (5.15 moles), in 250 ml of CH₂Cl₂ was then added dropwise at a rate which kept the reaction temperature below 15° C. Following the addition, the mixture was warmed to room temperature and stirred 2 hours. The reaction mixture was diluted with 900 ml of CH₂Cl₂ and 500 g of ice, followed by the slow addition of 2500 ml of 2.2 N NaOH. After testing to insure the solution was basic, the product was transferred to a separatory funnel and the organic layer was removed and set aside as extract #1. The aqueous was then extracted with 3×1250 ml of CH₂Cl₂, keeping each extraction as a separate fraction. The four organic extracts were then washed successively with a single 1250 ml portion of 0.6 N NaOH beginning with fraction #1 and proceeding through fraction #4. This wash procedure was repeated a second time with a fresh 1250 ml portion of 0.6 N NaOH. The organic extracts were then combined and dried over Na₂SO₄. Filtration and evaporation of solvent to a constant weight gave 825 g of N-mono-t-BOC-1,3-diaminopropane which was used without further purification.

A solution of methacrylic anhydride, 806 g (5.23 moles), in 1020 ml of CHCl₃ was placed in a 12 liter Morton flask equipped with overhead stirrer and cooled on an ice bath. Phenothiazine, 60 mg, was added as an inhibitor, followed by the dropwise addition of N-mono-t-BOC-1,3-diaminopropane, 825 g (4.73 moles), in 825 ml of CHCl₃. The rate of addition was controlled to keep the reaction temperature below 10° C. at all times. After the addition was complete, the ice bath was removed and the mixture was left to stir overnight. The product was diluted with 2400 ml of water and transferred to a separatory funnel. After thorough mixing, the aqueous layer was removed and the organic layer was washed with 2400 ml of 2 N NaOH, insuring that the aqueous layer was basic. The organic layer was then dried over Na₂SO₄ and filtered to remove drying agent. A portion of the CHCl₃ solvent was removed under reduced pressure until the combined weight of the product and solvent was approximately 3000 g. The desired product was then precipitated by slow addition of 11.0 liters of hexane to the stirred CHCl₃ solution, followed by overnight storage at 4° C. The product was isolated by filtration and the solid was rinsed twice with a solvent combination of 900 ml of hexane and 150 ml of CHCl₃. Thorough drying of the solid gave 900 g of N-[N′-(t-butyloxycarbonyl)-3-aminopropyl]-methacrylamide, m.p. 85.8° C. by DSC. Analysis on an NMR spectrometer was consistent with the desired product: ¹H NMR (CDCl₃) amide NH's 6.30-6.80, 4.55-5.10 (m, 2H), vinyl protons 5.65, 5.20 (m, 2H), methylenes adjacent to N 2.90-3.45 (m, 4H), methyl 1.95 (m, 3H), remaining methylene 1.50-1.90 (m, 2H), and t-butyl 1.40 (s, 9H).

A 3-neck, 2 liter round bottom flask was equipped with an overhead stirrer and gas sparge tube. Methanol, 700 ml, was added to the flask and cooled on an ice bath. While stirring, HCl gas was bubbled into the solvent at a rate of approximately 5 liters/minute for a total of 40 minutes. The molarity of the final HCl/MeOH solution was determined to be 8.5 M by titration with 1 N NaOH using phenolphthalein as an indicator. The N-[N′-(t-butyloxycarbonyl)-3-aminopropyl]methacrylamide, 900 g (3.71 moles), was added to a 5 liter Morton flask equipped with an overhead stirrer and gas outlet adapter, followed by the addition of 1150 ml of methanol solvent. Some solids remained in the flask with this solvent volume. Phenothiazine, 30 mg, was added as an inhibitor, followed by the addition of 655 ml (5.57 moles) of the 8.5 M HCl/MeOH solution. The solids slowly dissolved with the evolution of gas but the reaction was not exothermic. The mixture was stirred overnight at room temperature to insure complete reaction. Any solids were then removed by filtration and an additional 30 mg of phenothiazine were added. The solvent was then stripped under reduced pressure and the resulting solid residue was azeotroped with 3×1000 ml of isopropanol with evaporation under reduced pressure. Finally, the product was dissolved in 2000 ml of refluxing isopropanol and 4000 ml of ethyl acetate were added slowly with stirring. The mixture was allowed to cool slowly and was stored at 4° C. overnight. The N-(3-aminopropyl)methacrylamide hydrochloride was isolated by filtration and was dried to constant weight, giving a yield of 630 g with a melting point of 124.7° C. by DSC. Analysis on an NMR spectrometer was consistent with the desired product: ¹H NMR (D₂O) vinyl protons 5.60, 5.30 (m, 2H), methylene adjacent to amide N 3.30 (t, 2H), methylene adjacent to amine N 2.95 (t, 2H), methyl 1.90 (m, 3H), and remaining methylene 1.65-2.10 (m, 2H). The final compound was stored for use in the preparation of a monomer used in the synthesis of photoactivatable polymers as described, for instance, in Example 3(c).

(c) Preparation of N-[3-(4-Benzoylbenzamido)propyl]methacrylamide (BBA-APMA)

APMA, 120.0 g (0.672 moles), prepared according to the general method described in Example 3(b), was added to a dry 2 liter, three-neck round bottom flask equipped with an overhead stirrer. Phenothiazine, 23-25 mg, was added as an inhibitor, followed by 800 ml of chloroform. The suspension was cooled below 10° C. on an ice bath and 172.5 g (0.705 moles) of BBA-Cl, prepared according to the general method described in Example 3(a), were added as a solid. Triethylamine, 207 ml (1.485 moles), in 50 ml of chloroform was then added dropwise over a 1-1.5 hour time period. The ice bath was removed and stirring at ambient temperature was continued for 2.5 hours. The product was then washed with 600 ml of 0.3 N HCl and 2×300 ml of 0.07 N HCl. After drying over sodium sulfate, the chloroform was removed under reduced pressure and the product was recrystallized twice from toluene/chloroform (4/1) using 23-25 mg of phenothiazine in each recrystallization to prevent polymerization. Typical yields of BBA-APMA were 90% with a melting point of 147-151° C. Analysis on an NMR spectrometer was consistent with the desired product: ¹H NMR (CDCl₃) aromatic protons 7.20-7.95 (m, 9H), amide NH 6.55 (broad t, 1H), vinyl protons 5.65, 5.25 (m, 2H), methylenes adjacent to amide N's 3.20-3.60 (m, 4H), methyl 1.95 (s, 3H), and remaining methylene 1.50-2.00 (m, 2H). The final compound was stored for use in the synthesis of photoactivatable polymers as described, for instance, in Example 3(e).

(d) Preparation of N-Succinimidyl 6-Maleimidohexanoate (MAL-EAC-NOS)

A functionalized monomer was prepared in the following manner, and was used as described in Example 3(e) to introduce activated ester groups on the backbone of a polymer. 6-Aminohexanoic acid, 100.0 g (0.762 moles), was dissolved in 300 ml of acetic acid in a three-neck, 3 liter flask equipped with an overhead stirrer and drying tube. Maleic anhydride, 78.5 g (0.801 moles), was dissolved in 200 ml of acetic acid and added to the 6-aminohexanoic acid solution. The mixture was stirred one hour while heating on a boiling water bath, resulting in the formation of a white solid. After cooling overnight at room temperature, the solid was collected by filtration and rinsed with 2×50 ml of hexane. After drying, the typical yield of the (Z)-4-oxo-5-aza-2-undecendioic acid was 158-165 g (90-95%) with a melting point of 160-165° C. Analysis on an NMR spectrometer was consistent with the desired product: ¹H NMR (DMSO-d₆) amide proton 8.65-9.05 (m, 1H), vinyl protons 6.10, 6.30 (d, 2H), methylene adjacent to nitrogen 2.85-3.25 (m, 2H), methylene adjacent to carbonyl 2.15 (t, 2H), and remaining methylenes 1.00-1.75 (m, 6H).

(Z)-4-Oxo-5-aza-2-undecendioic acid, 150.0 g (0.654 moles), acetic anhydride, 68 ml (73.5 g, 0.721 moles), and phenothiazine, 500 mg, were added to a 2 liter three-neck round bottom flask equipped with an overhead stirrer. Triethylamine, 91 ml (0.653 moles), and 600 ml of THF were added and the mixture was heated to reflux while stirring. After a total of 4 hours of reflux, the dark mixture was cooled to <60° C. and poured into a solution of 250 ml of 12 N HCl in 3 liters of water. The mixture was stirred 3 hours at room temperature and then was filtered through a Celite 545 pad to remove solids. The filtrate was extracted with 4×500 ml of chloroform and the combined extracts were dried over sodium sulfate. After adding 15 mg of phenothiazine to prevent polymerization, the solvent was removed under reduced pressure. The 6-maleimidohexanoic acid was recrystallized from hexane/chloroform (2/1)to give typical yields of 76-83 g (55-60%) with a melting point of 81-85° C. Analysis on a NMR spectrometer was consistent with the desired product: ¹H NMR (CDCl₃) maleimide protons 6.55 (s, 2H), methylene adjacent to nitrogen 3.40 (t, 2H), methylene adjacent to carbonyl 2.30 (t, 2H), and remaining methylenes 1.05-1.85 (m, 6H).

The 6-maleimidohexanoic acid, 20.0 g (94.7 mmol), was dissolved in 100 ml of chloroform under an argon atmosphere, followed by the addition of 41 ml (0.47 mol) of oxalyl chloride. After stirring for 2 hours at room temperature, the solvent was removed under reduced pressure with 4×25 ml of additional chloroform used to remove the last of the excess oxalyl chloride. The acid chloride was dissolved in 100 ml of chloroform, followed by the addition of 12.0 g (0.104 mol) of N-hydroxysuccinimide and 16.0 ml (0.114 mol) of triethylamine. After stirring overnight at room temperature, the product was washed with 4×100 ml of water and dried over sodium sulfate. Removal of solvent gave 24.0 g of product (82%) which was used without further purification. Analysis on an NMR spectrometer was consistent with the desired product: ¹H NMR (CDCl₃) maleimide protons 6.60 (s, 2H), methylene adjacent to nitrogen 3.45 (t, 2H), succinimidyl protons 2.80 (s, 4H), methylene adjacent to carbonyl 2.55 (t, 2H), and remaining methylenes 1.15-2.00 (m, 6H). The final compound was stored for use in the synthesis of photoactivatable polymers as described, for instance, in Example 3(e).

(e) Preparation of a Copolymer of Acrylamide, BBA-APMA, and MAL-EAC-NOS

A photoactivatable copolymer of the present invention was prepared in the following manner. Acrylamide, 3.849 g (54.1 mmol), was dissolved in 52.9 ml of tetrahydrofuran (THF), followed by 0.213 g ( 0.61 mmol) of BBA-APMA, prepared according to the general method described in Example 3(c), 0.938 g (3.04 mmol) of MAL-EAC-NOS, prepared according to the general method described in Example 3(d), 0.053 ml (0.35 mmol) of N,N,N′,N′-tetramethylethylenediamine (TEMED), and 0.142 g (0.86 mmol) of 2,2′-azobisisobutyronitrile (AIBN). The solution was deoxygenated with a helium sparge for 4 minutes, followed by an argon sparge for an additional 4 minutes. The sealed vessel was then heated overnight at 55° C. to complete the polymerization. The solid product was isolated by filtration and the filter cake was rinsed thoroughly with THF and CHCl₃. The product was dried in a vacuum oven at 30° C. to give 5.234 g of a white solid. NMR analysis (DMSO-d₆) confirmed the presence of the NOS group at 2.75 ppm and the photogroup load was determined to be 0.104 mmol BBA/g of polymer. MAL-EAC-NOS composed 5 mole % of the polymerizable monomers in this reaction.

(f) Preparation and Evaluation of a Photopolymer Derivatized with Oligonucleotides

A 40-mer probe 5′-GTCTGAGTCGGAGCC AGGGCGGCCGCCAACAGCAGGAGCA-3′ (ID4) was synthesized with an amine modification as described for ID1. Oligo amine-ID4, 40 μg (15 μl of 2.67 mg/ml stock in water) was incubated with 80 μg (80 μl of 1 mg/ml freshly made in water) of a copolymer of acrylamide, BBA-APMA, and MAL-EAC-NOS, prepared as described in Example 3(e), and 305 μl of incubation buffer. The reaction mixture was stirred at room temperature for 2 hours. The resulting photopoly-ID4 was used without further purification for immobilization.

Amine-ID4 and photopoly-ID4 at 10 pmole oligo/0.1 ml per well were incubated in PP and poly(vinyl chloride) microwell plates (PVC, Dynatech, Chantilly, Va.) in 50 mM phosphate buffer, pH 8.5, 1 mM EDTA for 1.5 hours at 37° C. The plates were illuminated or adsorbed as described in Example 1(c). Hybridization was performed as described in Example 1(c) using the complementary ID3 detection oligonucleotide or non-complementary ID2 oligonucleotide. The results from Table 3 indicate that the illuminated photopoly-oligonucleotide had 13- and 2-fold higher hybridization signals than the adsorbed control on PP and PVC surfaces, respectively. In contrast, illumination did not have a useful effect on amine-ID4 immobilization.

TABLE 3
Hybridization signals (A₆₅₅ ± standard
deviation) from amine-ID4 and photopoly-ID4 on
PP and PVC microwell plates.

| | |
|---|---|
| Adsorbed Control | Illuminated |

| | | Non- | | Non- |
|---|---|---|---|---|
| | Complem. | complem. | Complem. | complem. |
| | Det. | Det. | Det. | Det. |
| | ID3 | ID2 | ID3 | ID2 |
| PP plates | | | | |
| Amine-ID4 | 0.034 ± | 0.011 ± | 0.001 ± | 0.014 ± |
| | 0.034 | 0.001 | 0.002 | 0.005 |
| Photopoly-ID4 | 0.099 ± | 0.017 ± | 1.356 ± | 0.019 ± |
| | 0.033 | 0.015 | 0.078 | 0.021 |
| PVC plates | | | | |
| Amine-ID4 | 0.153 ± | 0.087 ± | 0.001 ± | 0.046 ± |
| | 0.031 | 0.025 | 0.002 | 0.006 |
| Photopoly-ID4 | 0.992 ± | 0.097 ± | 1.356 ± | 0.087 ± |
| | 0.071 | 0.013 | 0.078 | 0.071 |

### Example 4

### Preparation of a Benzophenone Labeled Oligonucleotide by Direct Synthesis

(a) Preparation of 4-Bromomethylbenzophenone (BMBP)

4-Methylbenzophenone, 750 g (3.82 moles), is added to a 5 liter Morton flask equipped with an overhead stirrer and dissolved in 2850 ml of benzene. The solution is then heated to reflux, followed by the dropwise addition of 610 g (3.82 moles) of bromine in 330 ml of benzene. The addition rate is approximately 1.5 ml/min and the flask is illuminated with a 90 watt (90 joule/sec) halogen spotlight to initiate the reaction. A timer is used with the lamp to provide a 10% duty cycle (on 5 seconds, off 40 seconds), followed in one hour by a 20% duty cycle (on 10 seconds, off 40 seconds). After cooling, the reaction mixture is washed with 10 g of sodium bisulfite in 100 ml of water, followed by washing with 3×200 ml of water. The product is dried over sodium sulfate and recrystallized twice from toluene/hexane (1/3). The final compound is stored for use in the preparation of a reagent suitable for derivatization of nucleic acids as described in Example 4(b).

(b) Preparation of 4-Benzoylbenzylether-C₁₂-phosphoramidite

1,12-Dodecanediol, 5.0 g (24.7 mmol), is dissolved in 50 ml of anhydrous THF in a dry flask under nitrogen. The sodium hydride, 0.494 g of a 60% dispersion in mineral oil (12.4 mmol), is added in portions over a five minute period. The resulting mixture is stirred at room temperature for one hour. BMBP, 3.40 g (12.4 mmol), prepared according to the general method described in Example 4(a), is added as a solid along with sodium iodide (0.185 g, 1.23 mmol) and tetra-n-butylammonium bromide (0.398 g, 1.23 mmol). The mixture is stirred at a gentle reflux for 24 hours. The reaction is then cooled, quenched with water, acidified with 5% HCl, and extracted with chloroform. The organic extracts are dried over sodium sulfate and the solvent is removed under vacuum. The product is purified on a silica gel flash chromatography column using chloroform to elute non-polar impurities, followed by elution of the product with 80:20 chloroform: ethyl acetate. Pooling of appropriate fractions provides the desired compound after removal of solvent under reduced pressure.

The ether product from above, 0.100 g (0.252 mmol), is dissolved in chloroform under an argon atmosphere. N,N-Diisopropylethylamine, 0.130 g (1.00 mmol), is added and the temperature is adjusted to 0° C. using an ice bath. 2-Cyanoethyl diisopropylchlorophosphoramidite, 0.179 g (0.756 mmol), is then added in three equal portions over about 10 minutes. Stirring is continued for a total of three hours, after which time the reaction is quenched with 5% NaHCO₃ and diluted with 5 ml of chloroform. The organic layer is separated, dried over sodium sulfate, and evaporated to provide a residual oil. The crude product is purified on a silica gel flash chromatography column using a 5% methanol in chloroform solvent, followed by a ammonium hydroxide/methanol/chloroform (0.5/2.5/7) solvent system. The appropriate fractions are pooled and the solvent is removed to provide the desired product, suitable for derivatization of a nucleic acid.

(c) Preparation of a Benzophenone Labeled Oligonucleotide

A 30-mer oligonucleotide is synthesized on silica beads using standard oligonucleotide procedures and the beads are placed in a sealed vessel under an argon atmosphere. Solutions of 12.5 mg (22 μmol) of the phosphoramidite prepared in Example 4(b) in 0.5 ml of chloroform and 5 mg (71 μmol) of tetrazole in 0.5 ml of acetonitrile are then added. The mixture is gently agitated for 1 hour, followed by the removal of the supernatant. The beads are washed with chloroform, acetonitrile, and methylene chloride, followed by oxidation for 5 minutes with 1.5 ml of a 0.1 M iodine solution in THF/pyridine/water (40/20/1). After removal of this solution, the beads are washed with methylene chloride and dried with an argon stream. Concentrated ammonium hydroxide is then added to the beads and they are allowed to stand for 1 hour at room temperature. The ammonium hydroxide solution is then removed and the beads are rinsed with an additional 1 ml of ammonium hydroxide. The combined solution extracts are then stored at 55° C. overnight, followed by lyophilization to isolate the photolabeled oligonucleotide.

What is claimed is:

1. A method for generating a microarray, comprising:
(a) applying at least one reagent solution containing receptor molecules to a solid support to form a first applied spot pattern, wherein spots in the first applied spot pattern have an area and wherein the reagent solution, the receptor molecules, the solid support, or any combination thereof includes at least one photoreactive group;
(b) illuminating the first applied spot pattern to immobilize the receptor molecules to the solid support in a first immobilized spot pattern, wherein spots in the first immobilized spot pattern have an area and wherein the area of the spots in the first immobilized spot pattern is less than the area of the spots in the first applied spot pattern.

2. The method according to claim 1, wherein the step of applying comprises printing.

3. The method according to claim 1, wherein the step of illuminating comprises masked illumination.

4. The method according to claim 1, wherein the step of illuminating comprises mirrored laser illumination.

5. The method according to claim 1, wherein the receptor molecule includes at least one photoreactive group.

6. The method according to claim 1, wherein the solid support includes at least one photoreactive group.

7. The method according to claim 1, wherein the spots of the first applied spot pattern have a center to center distance and the spots of the first immobilized spot pattern have a center to center distance and the center to center distances of the first applied spot pattern and the first immobilized spot pattern are the same.

8. The method according to claim 1, further comprising a washing step after the step of illuminating.

9. The method according to claim 1, further comprising a step of:
(a) applying at least one reagent solution containing receptor molecules to the solid support to form a second applied spot pattern, wherein spots in the second applied spot pattern have an area and wherein the reagent solution, the receptor molecules, the solid support, or any combination thereof include at least one photoreactive group; and
(b) illuminating the second applied spot pattern to immobilize the receptor molecules to the solid support to form a second immobilized spot pattern wherein spots in the second immobilized spot pattern have an area, and the area of the spots in the second immobilized spot pattern is less than the are of the spots in the second applied spot pattern and the spots in the second immobilized spot pattern are offset from the spots of the first immobilized spot pattern.

10. The method according to claim 9, further comprising repeating steps of:
(a) applying at least one reagent solution containing receptor molecules to the solid support to form an applied spot pattern, wherein the spots in the applied spot pattern have an area and wherein the reagent solution, the receptor molecules, the solid support, or any combination thereof include at least one photoreactive group; and
(b) illuminating the applied spot pattern to immobilize the receptor molecules to the solid support in a immobilized spot pattern wherein spots in the immobilized spot pattern have an area, and the area of the spots in the immobilized spot pattern is less than the area of the spots in the applied spot pattern and the spots in the immobilized spot pattern are offset from an existing immobilized spot pattern,
wherein repeating steps (a) and (b) is used to form a high density array.

11. The method according to claim 9, wherein the first immobilized spot pattern has a pitch and the second immobilized spot pattern has a pitch and the pitch of the second immobilized spot pattern is the same as the pitch of the first immobilized spot pattern.

12. The method according to claim 1, wherein the step of illuminating comprises illuminating the first applied spot pattern in a circular configuration.

13. The method according to claim 1, wherein the step of illuminating comprises illuminating the first applied spot pattern in a non-circular configuration.

14. The method according to claim 1, further comprising a step of:
(a) applying at least one reagent solution containing receptor molecules to the solid support to form a second applied spot pattern, wherein spots in the second applied spot pattern have an area and wherein the reagent solution, the receptor molecules, the solid support, or any combination thereof includes at least one photoreactive group;
(b) illuminating the second applied spot pattern in a different configuration than the first immobilized spot pattern to immobilize the receptor molecules to the solid support in a second immobilized spot pattern having a different configuration than the first immobilized spot pattern wherein spots in the second immobilized spot pattern have an area, and the area of the spots in the second immobilized spot pattern is less than the area of the spots in the second applied spot pattern.

15. The method according to claim 14, wherein the second immobilized spot pattern is offset from the first immobilized spot pattern.

16. The method according to claim 14, wherein the second immobilized spot pattern is superimposed on the first immobilized spot pattern.

17. A microarray prepared by the method of claim 1.

18. A microarry prepared by the method of claim 10.

19. A microarray prepared by the method of claim 14.

20. A microarray comprising a solid support having a pattern of nucleic acid spots wherein the spots have a diameter of less than 100 μm and comprise nucleic acids having a sequence of at least 30 bases.

21. The microarray according to claim 20, wherein the nucleic acids have a sequence of at least 40 bases.

22. The microarray according to claim 20, wherein the nucleic acids have a sequence of at least 50 bases.

23. The microarray according to claim 20, wherein the nucleic acids comprise cDNA.

24. The microarray according to claim 20, wherein the nucleic acid spots have a diameter of less than 50 μm.

25. The microarray according to claim 20, wherein the pattern of nucleic acid spots has a density of more than 5,000 spots per square centimeter.

26. The microarray according to claim 20, wherein the pattern of nucleic acid spots has a density between 10,000 and 100,000 spots per square centimeter.

27. The microarray according to claim 20, wherein the spots have an essentially circular configuration.

28. The microarray according to claim 20, wherein the spots have a non-circular configuration.

29. The microarray according to claim 20, comprising spots having differing configurations.

30. The microarray according to claim 29, wherein the spots having differing configurations are offset from one another.

31. The microarray according to claim 29, wherein the spots having differing configurations are superimposed on one another.

32. The microarray according to claim 20, wherein the solid support comprises a two-dimensional solid support.

33. The microarray according to claim 20, wherein the solid support comprises a three-dimensional solid support.
